(19) United States
(12) Patent Application Publication
(10) Pub. No.: US 2026/0256620 A1
(43) Pub. Date: Sep. 3, 2026

(54) EYE PROTECTIVE WATERPROOF PATCH

(71) Applicant: Da Hyun KO, Seoul (KR)

(72) Inventor: Da Hyun KO, Seoul (KR)

(21) Appl. No.: 18/870,839

(22) PCT Filed: Jun. 20, 2022
(86) PCT No.: PCT/KR2022/008680

§ 371 Date: Dec. 2, 2024

(30) Foreign Application Priority Data
Jun. 7, 2022 (KR) 10-2022-0068682

(51) Int. Cl. A61F 9/02

(52) U.S. Cl. CPC A61F 9/026

(57) ABSTRACT

The present invention relates to an eye protective waterproof patch, which is an improvement of “an eye protective cap” of Korean utility model registration No. KR 20-0487330 by the present applicant, and comprises: a lens 110 having an edge portion 112 formed on the circumference of a main body 111; an adhesive film 120 having a hole circumference portion 123 joined to the edge portion 112 of the lens 110; a backing sheet 130 attached to the rear surface of the adhesive film 120; and a front release sheet 140 attached to the front surface of the adhesive film 120, wherein, when the edge portion 112 of the lens 110 is adhered to the hole circumference portion 123 of the adhesive film 120, an outer surface 112a of the edge portion 112 is adhered to an inner adhesive surface 123a of the hole circumference portion 123.

### DESCRIPTION

### Technical Field

The present invention relates to an eye protective waterproof patch, which is an improvement of “an eye protective cap” of Korean Utility Model Registration No. KR 20-0487330 by the present applicant, and more particularly, to an eye protective waterproof patch, in which the edge portion of the lens is adhered to the hole circumference portion of the adhesive film, and thus the outer surface of the edge portion is attached to the inner adhesive surface of the hole circumference portion.

### Background

An “eye protective cap” is introduced in Korean Utility Model Registration No. KR 20-0487330 (registered on Aug. 29, 2018).

The eye protective cap includes a base cover; an attachment member attached to one surface of the base cover and formed so that a cap portion protrudes in one direction; and a front cover attached to one surface of the attachment member and having a passage hole formed so that the cap portion passes through, wherein the attachment member includes an adhesive film, a protective cover attached to the periphery of one side surface of the adhesive film, and a cap portion attached to the center of the other surface of the adhesive film, and the cap portion includes a base portion attached to the other surface of the adhesive film, and a protective cap protrusively formed in one direction from the center of the base portion.

Referring to FIGS. 1 and 2, however, in the eye protective cap, the outer surface 21a of the hole circumference portion formed in the adhesive film 20 is joined to the inner surface 11a of the base portion 11 formed on the circumference of the cap portion 10, whereby, as shown in FIG. 2, when attached around the user's eyes, a flexible soft adhesive film 20 is attached around the eyes, and the base portion 11 of the cap portion 10 is disposed thereon. Consequently, there is a disadvantage in that when muscles around the eyes move or an external force is applied to the cap portion 10, the base portion 11 of the cap portion 10 is easily detached from the hole circumference portion 21 of the adhesive film 20, and moisture may flow in between the inner surface 11a of the base portion 11 and the outer surface 21a of the hole circumference portion 21.

### DETAILED DESCRIPTION OF THE INVENTION

### Technical Problem

Therefore, it is an object of the present invention to provide an eye protective waterproof patch, in which when the edge portion of the lens is adhered to the hole circumference portion of the adhesive film, and the outer surface of the edge is adhered to the adhesive surface on the inside of the hole circumference portion, whereby when the adhesive film is attached around a user's eyes, the inner side surface of the edge portion comes into direct contact with the user's eyes, and the adhesive surface of the hole circumference portion is adhered to the outer surface of the edge portion, so that even if facial muscles around the eyes move or external force is applied to the lens, the lens is not separated from the adhesive film, and moisture can be prevented from flowing in between the edge portion of the lens and the hole circumference portion of the adhesive film.

It is another object of the present invention to provide an eye protective waterproof patch, in which the edge portions of the lens are curved left and right to correspond to the curved surface around the eye, whereby the edge portion adheres better to the eye than a flat edge portion, and when the adhesive film is adhered to the eye, stress does not occur at the edges, so the left and right sides of the edge portions can be prevented from being separated from the adhesive film due to stress.

### Technical Solution

In order to achieve the above object, according to an embodiment of the present disclosure, there is provided an eye protective waterproof patch, comprising: a lens having an edge portion formed on the circumference of a main body; an adhesive film having a hole circumference portion joined to the edge portion of the lens; a backing sheet attached to the rear surface of the adhesive film; and a front release sheet attached to the front surface of the adhesive film, wherein, when the edge portion of the lens is adhered to the hole circumference portion of the adhesive film, an outer surface of the edge portion is adhered to an inner adhesive surface of the hole circumference portion.

When the adhesive film is adhered around the user's eyes so that the lens covers the user's eyes, the inner surface of the edge portion of the lens comes into direct contact with the user's eyes, and the inner adhesive surface of the hole circumference portion is disposed on the outer surface of the edge.

The edge portion 112 has a width W1 of 3 mm±1 mm.

The lens 110 is configured such that a main body 111 and an edge portion 112 are curved left and right to correspond to the curved surface around the eye.

The edge portion 112 has a radius of curvature R1 of 48 mm±1 mm.

The lens 110 has a left-right width S1 of 50 mm, a height h1 of 40 mm, and a left-right radius of curvature R2 of the main body 111 of 26 mm±1 mm.

The adhesive film is made of a soft transparent polyurethane material, is formed with an oval-shaped hole corresponding to the lens, and is applied with an adhesive onto one surface.

The backing sheet is configured such that the side end portion protrudes by 3 mm to 4 mm further to the side than the side end portion of the front release sheet, a hole corresponding to the lens main body is formed in the center, a release portion is formed around the hole, and a cutting line is formed in the center of the release portion.

Thereby, when the release portion of the backing sheet is separated from the backing sheet in the state where the adhesive film is disposed between the front release sheet and the backing sheet, the hole circumference portion of the adhesive film is exposed inside the hole of the backing sheet, whereby after the main body of the lens passes through the hole of the adhesive film, the edge portion of the lens is joined to the hole circumference portion of the adhesive film.

The lens further comprises an upper wing portion and a lower wing portion formed at the edge portion, wherein the upper wing portion and the lower wing portion are disposed facing each other vertically around the main body of the lens.

The upper wing portion is formed so that the width W3 is smaller than the width W4 of the lower wing portion, and the height h3 is 2 to 2.5 times higher than the height h4 of the lower wing portion.

The lens is formed in a water drop shape with its main body lying down.

### Advantageous Effects

Therefore, an eye protective waterproof patch according to the present invention has the following effects; when the adhesive film is attached around a user's eyes, the inner side surface of the edge portion comes into direct contact with the user's eyes, and the adhesive surface of the hole circumference portion is disposed on the outer surface of the edge portion, so that even if facial muscles around the eyes move or external force is applied to the lens, the lens does not separate from the adhesive film, and moisture can be prevented from flowing in between the edge portion of the lens and the hole circumference portion of the adhesive film, and also the edge portions of the lens are curved left and right to correspond to the curved surface around the eye, whereby the edge portion adheres better to the eye than a flat edge portion, and when the adhesive film is adhered to the eye, stress does not occur at the edges, so the left and right sides of the edge portions can be prevented from being separated from the adhesive film due to stress.

In addition, an eye protective waterproof patch according to the present invention includes the properties and effects mentioned in the prior art, such as portability, ease of use, hygiene, waterproofing and anti-fog properties, anti-viral coating, and the like.

### BRIEF DESCRIPTION OF THE DRAWINGS

FIG. 1 is a cross-sectional view and a partially enlarged detailed view showing a conventional eye protective cap

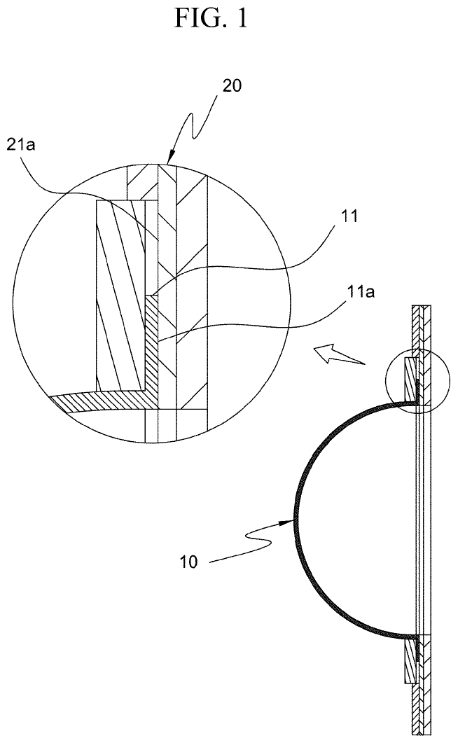

FIG. 2 is a cross-sectional view and a partially enlarged detailed view showing a state where the eye protective cap of FIG. 1 is adhered around the eye

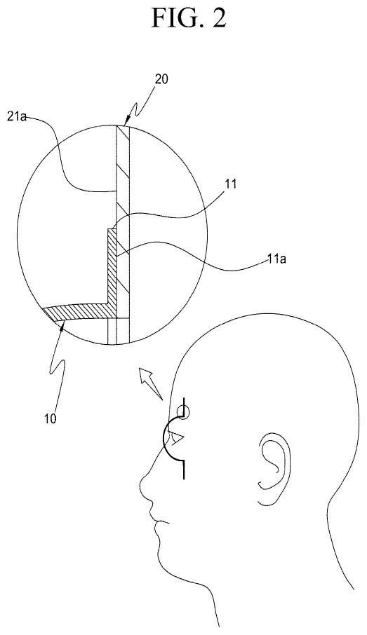

FIG. 3 is a perspective view showing an eye protective waterproof patch according to the present invention.

FIG. 4 is an exploded perspective view showing an eye protective waterproof patch according to the present invention

FIG. 5 is a cross-sectional view and a partially enlarged detailed view along the line A-A of FIG. 3 showing an eye protective waterproof patch according to the present invention adhered around the eye

FIG. 6 is a cross-sectional view and a partially enlarged detailed view showing a state where an eye protective waterproof patch according to the present invention is adhered around the eye.

FIG. 7 is a front view showing an eye protective waterproof patch according to the present invention.

FIG. 8 is an exploded perspective view showing an example in which the edge portion of the lens is joined to the hole circumference portion of the adhesive film after the release portion is separated from the backing sheet.

FIG. 9 is a front view showing an eye protective waterproof patch according to the present invention with a different lens shape.

FIG. 10 is a front view showing an eye protective waterproof patch according to the present invention with a different lens shape.

### DETAILED DESCRIPTION OF THE EMBODIMENTS

Hereinafter, embodiments according to the present invention will be described in detail with reference to the accompanying drawings. However, this description does not limit the present invention within specific embodiments and it should be understood that the present invention covers all the modifications, equivalents, and replacements within the idea and technical scope of the present invention. In description of the drawings, like reference numerals in the drawings denote like elements. Unless otherwise defined, all terms (including technical and scientific terms) used herein have the same meaning as commonly understood by one having ordinary skill in the art to which this invention belongs.

Throughout the descriptions herein, when a certain part “includes” a component, this does not indicate that the certain part excludes other components, but indicates that the part may further include other components, unless otherwise defined. The term “inside” refers to a location that is relatively closer to the skin around the user's eye to which the adhesive film is adhered in a particular component than the “outside”, and conversely, the term “outside” refers to a location that is relatively farther from the skin around the user's eye to which the adhesive film is adhered in a particular component than the “inside”.

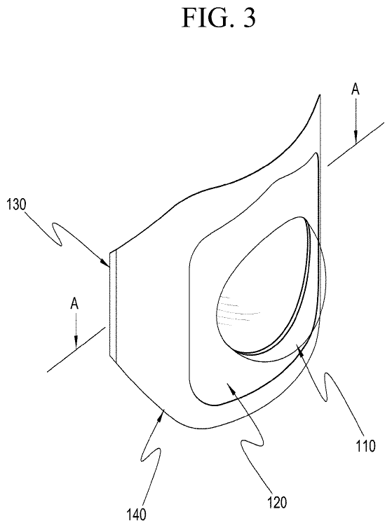

Referring to FIGS. 3 to 5, the eye protective waterproof patch according to the present disclosure comprises a lens 110 having an edge portion 112 formed on the circumference of a main body 111; an adhesive film 120 having a hole circumference portion 123 joined to the edge portion 112 of the lens 110; a backing sheet 130 attached to the rear surface of the adhesive film 120; and a front release sheet 140 attached to the front surface of the adhesive film 120.

Here, the eye protective waterproof patch according to the present disclosure is characterized in that, when the edge portion 112 of the lens 110 is adhered to the hole circumference portion 123 of the adhesive film 120, an outer surface 112a of the edge portion 112 is adhered to an inner adhesive surface 123a of the hole circumference portion 123.

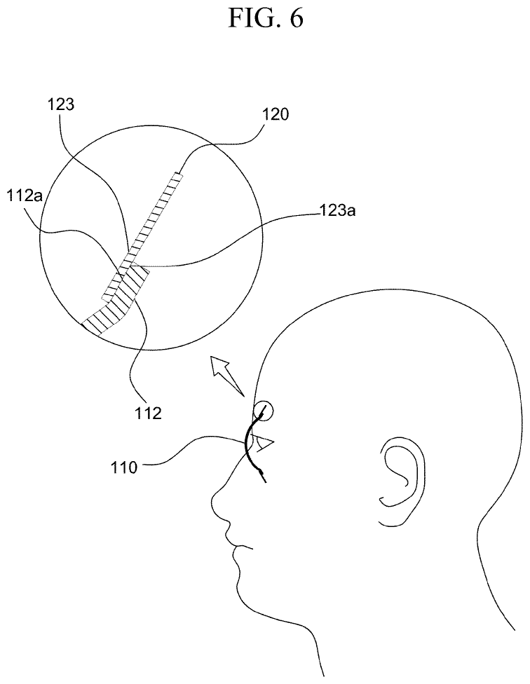

Thereby, as shown in FIG. 6, when the adhesive film 120 is adhered around the user's eyes so that the lens 110 covers the user's eyes 160, the inner surface 112b of the edge portion 112 of the lens 110 comes into direct contact with the user's eyes, and the inner adhesive surface 123a of the hole circumference portion 123 is disposed on the outer surface 112a of the edge portion 112, whereby even if the facial muscles around the eyes move or an external force is applied to the lens 110, the edge portion 112 of the lens 110 does not separate from the hole circumference portion 123 of the adhesive film 120. In addition, since the outer surface 112a of the edge portion 112 is adhered to the inner adhesive surface 123a of the hole circumference portion 123, there is an advantage in that moisture can be prevented from flowing between the edge portion 112 of the lens 110 and the hole circumference portion 123 of the adhesive film 120.

The edge portion 112 has a width W1 of 3 mm±1 mm. Generally, having a width of 3 mm may be appropriate for minimizing the area that comes into contact with the user's face and also maintaining adhesion with the adhesive film 120, but the above-mentioned error may occur during the manufacturing process.

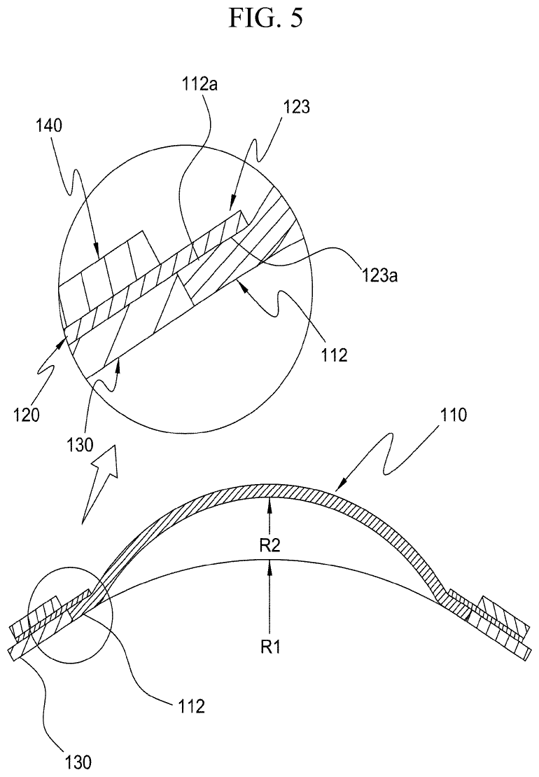

Referring again to FIG. 5, the lens 110 has a main body 111 and an edge portion 112 that are curved left and right to correspond to the curved surface around the eye. At this time, the edge portion 112 has a radius of curvature R1 of 48 mm±1 mm.

Thereby, the curved edge portion 112 closely adheres better to the eye area than the flat edge portion, and when the adhesive film 120 is adhered to the eye area, stress is not generated in the circumference portion 112, so that the left and right sides of the circumference portion 112 can be prevented from being separated from the hole circumference portion 123 of the adhesive film 120 due to stress.

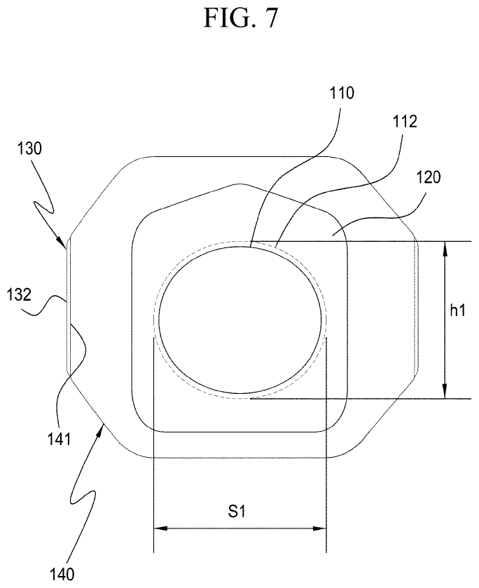

Referring to FIG. 7, the lens 110 has a left and right width S1 of 50 mm, a height h1 of 40 mm, and a left and right radius of curvature R2 (see FIG. 5) of the main body 111 of 26 mm±1 mm, which is formed larger than that of a conventional lens.

In this way, in the present invention, the lens 110 is formed to be large enough to cover the eye, so that the edge portion of the lens 110 does not cause inconvenience such as being placed on the eyelid when worn by a user.

In addition, the lens 110 has the same composition and physical and chemical properties as the cap introduced in the “Eye Protective Cap” of Korean Utility Model Registration No. 20-0487330 by the present applicant.

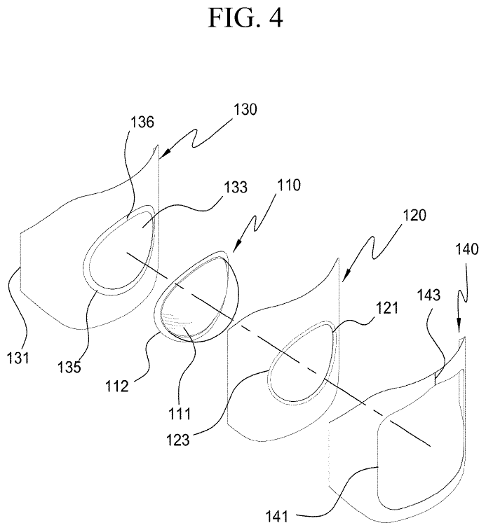

The adhesive film 120 is made of a soft transparent polyurethane material, and is formed with an oval-shaped hole 121 (see FIG. 4) corresponding to the lens, and is applied with an adhesive onto one surface.

Referring to FIGS. 4 and 7, the side end portion 132 of the backing sheet 130 protrudes by 3 mm to 4 mm further to the side than the side end portion 141 of the front release sheet 140, a hole 133 corresponding to the lens main body 111 is formed in the center, a release portion 135 is formed around the hole 133, and a cutting line 136 is formed in the center of the release portion 135.

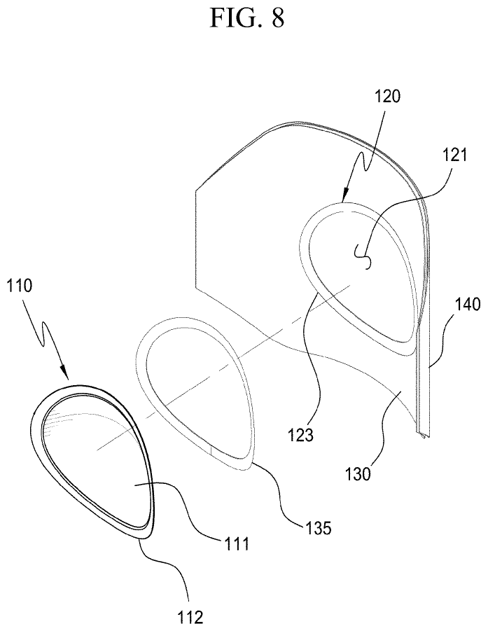

Referring to FIG. 8, when the release portion 135 of the backing sheet 130 is separated from the backing sheet 130 in the state where the adhesive film 120 is disposed between the front release sheet 140 and the backing sheet 130, the hole circumference portion 123 of the adhesive film 120 is exposed inside the hole 133 of the backing sheet 130, whereby after the main body 111 of the lens 110 passes through the hole 121 of the adhesive film 120, the edge portion 112 of the lens 110 is joined to the hole circumference portion 123 of the adhesive film 120.

As shown in FIG. 4, a release portion 135 is formed on the backing sheet 130, and therefore, there is an advantage in that as shown in FIG. 8, the release portion 135 is separated, and then the edge portion 112 of the lens 110 can be easily adhered to the hole circumference portion 123 of the adhesive film 120. In addition, the side end portion 131 of the backing sheet 130 protrudes by 3 to 4 mm to the side from the side end portion 141 of the front release sheet 140, so that when a user uses the backing sheet 130, the backing sheet 130 can be easily separated from the adhesive film 120 by taking the protruding portion.

Referring again to FIG. 4, the front release sheet 140 is finally separated after the adhesive film is attached around the user's eyes, a pentagonal-shaped hole 141 is formed in the center, and a cutting line 143 is formed in the upper center.

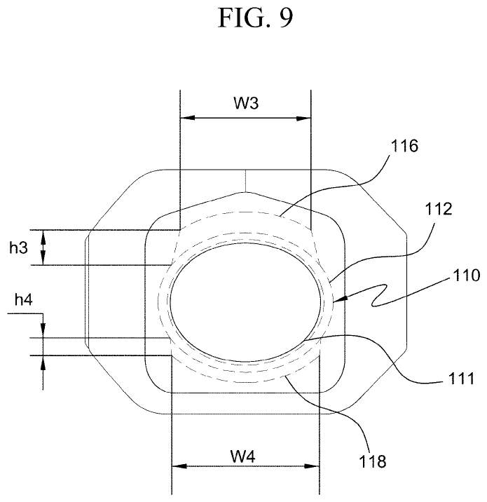

Referring to FIG. 9, the lens 110 further includes an upper wing portion 116 and a lower wing portion 118 formed on the edge portion 112.

The upper wing portion 116 and the lower wing portion 118 are disposed facing each other vertically with the main body 111 of the lens 110 as the center. The upper wing portion 116 is formed so that the left-right width W3 is smaller than the left-right width W4 of the lower wing portion 118, and the height h3 is formed 2 to 2.5 times higher than the height h4 of the lower wing portion 118.

As a result, when the adhesive film is applied around the user's eyes, the upper wing portion 116 of the lens 110 as well as the edge portion 112 of the lens 110 are in close contact with the eyebrows, and the lower wing portion 118 is in close contact with the lower portion of the eye, thereby increasing the adhesion of the lens and improving the user's sense of stability.

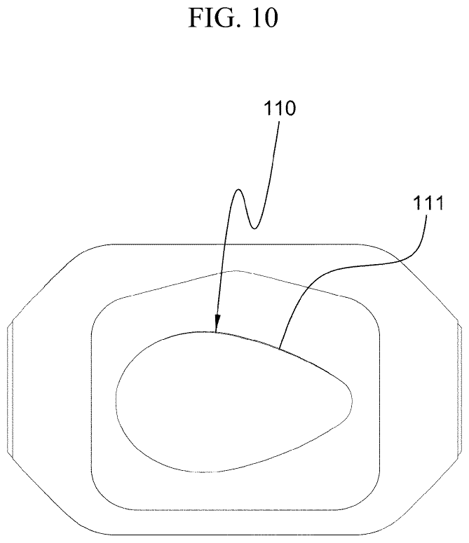

Referring to FIG. 10, as another alternative, the lens 110 is formed in a water drop shape with the main body 111 lying down.

The water drop shape of the lens 110 is less susceptible to positional changes, such as wrinkles on the nose, when the lens is disposed around the eye, and is therefore more advantageous in terms of ensuring the user's field of vision.

Although the present invention has been described above based on specific details such as specific components and limited embodiments and drawings, this is merely provided to aid in a more comprehensive understanding of the present invention, and the present invention is not limited to the above embodiments, and those with ordinary knowledge in the field to which the present invention belongs can make various modifications and variations from these descriptions.

Therefore, the spirit and idea of the present invention should not be limited to the above embodiments, and it will be understood by those skilled in the art that not only the scope of the claims described below, but also all modifications equivalent to or equivalent to the scope of the claims belong to the scope of the sprit and idea of the present invention.

1. An eye protective waterproof patch, comprising:
a lens 110 having an edge portion 112 formed on the circumference of a main body 111; an adhesive film 120 having a hole circumference portion 123 joined to the edge portion 112 of the lens 110; a backing sheet 130 attached to the rear surface of the adhesive film 120; and a front release sheet 140 attached to the front surface of the adhesive film 120,
wherein, when the edge portion 112 of the lens 110 is adhered to the hole circumference portion 123 of the adhesive film 120, an outer surface 112a of the edge portion 112 is adhered to an inner adhesive surface 123a of the hole circumference portion 123.

2. The eye protective waterproof patch according to claim 1, wherein:
when the adhesive film 120 is adhered around the user's eyes so that the lens 110 covers the user's eyes 160, the inner surface 112b of the edge portion 112 of the lens 110 comes into direct contact with the user's eyes, and the inner adhesive surface 123a of the hole circumference portion 123 is disposed on the outer surface 112a of the edge portion 112.

3. The eye protective waterproof patch according to claim 1, wherein:
the edge portion 112 has a width W1 of 3 mm±1 mm.

4. The eye protective waterproof patch according to claim 1, wherein:
the lens 110 is configured such that a main body 111 and an edge portion 112 are curved left and right to correspond to the curved surface around the eye.

5. The eye protective waterproof patch according to claim 4, wherein:
the edge portion 112 has a radius of curvature R1 of 48 mm±1 mm.

6. The eye protective waterproof patch according to claim 1, wherein:
the lens 110 has a left-right width S1 of 50 mm, a height h1 of 40 mm, and a left-right radius of curvature R2 of the main body 111 of 26 mm±1 mm.

7. The eye protective waterproof patch according to claim 1, wherein:
the adhesive film 120 is made of a soft transparent polyurethane material, is formed with an oval-shaped hole 121 corresponding to the lens, and is applied with an adhesive onto one surface.

8. The eye protective waterproof patch according to claim 1, wherein:
the backing sheet 130 is configured such that the side end portion 132 protrudes by 3 mm to 4 mm further to the side than the side end portion 141 of the front release sheet 140, a hole 133 corresponding to the lens main body 111 is formed in the center, a release portion 135 is formed around the hole 133, and a cutting line 136 is formed in the center of the release portion 135.

9. The eye protective waterproof patch according to claim 1, wherein:
when the release portion 135 of the backing sheet 130 is separated from the backing sheet 130 in the state where the adhesive film 120 is disposed between the front release sheet 140 and the backing sheet 130, the hole circumference portion 123 of the adhesive film 120 is exposed inside the hole 133 of the backing sheet 130, whereby after the main body 111 of the lens 110 passes through the hole 121 of the adhesive film 120, the edge portion 112 of the lens 110 is joined to the hole circumference portion 123 of the adhesive film 120.

10. The eye protective waterproof patch according to claim 1, wherein:
the lens 110 further comprises an upper wing portion 116 and a lower wing portion 118 formed at the edge portion 112, wherein the upper wing portion 116 and the lower wing portion 118 are disposed facing each other vertically around the main body 111 of the lens 110.

11. The eye protective waterproof patch according to claim 10, wherein:
the upper wing portion 116 is formed so that the width W3 is smaller than the width W4 of the lower wing portion 118, and the height h3 is 2 to 2.5 times higher than the height h4 of the lower wing portion 118.

12. The eye protective waterproof patch according to claim 1, wherein:
the lens 110 is formed in a water drop shape with its main body lying down.
